(19) United States
(12) Patent Application Publication
(10) Pub. No.: US 2016/0354781 A1
(43) Pub. Date: Dec. 8, 2016

(54) MICROFLUIDIC PLATE FOR SAMPLE PROCESSING

(71) Applicant: National Taiwan University, Taipei (TW)

(72) Inventors: Andrew Man Chung Wo, Taipei (TW); Lo-Chang Hsiung, Taipei (TW); Wei-Yuan Ma, Taipei (TW)

(21) Appl. No.: 15/097,259

(22) Filed: Apr. 12, 2016

Related U.S. Application Data
(60) Provisional application No. 62/172,294, filed on Jun. 8, 2015.

(51) Int. Cl. B01L 3/00; G01N 33/50

(52) U.S. Cl. CPC B01L 3/502715; B01L 3/502761; G01N 33/5011; B01L 2300/0861; B01L 2300/0803; B01L 2200/0668

(57) ABSTRACT

The invention provides a microfluidic plate for various biological experiments and analyses. The microfluidic plate of the invention comprises one or more unit formed on or in the plate, wherein the unit comprises (a) a chamber; (b) a fluid channel adjacent to the chamber; (c) at least one microfluidic gap region connected between the chamber and the channel and in fluidic communication therewith; and (d) at least one opening located at the channel and connected with the gaps and in fluidic communication with the gaps.

### RELATED APPLICATIONS

This application claims the benefit of U.S. Provisional Application No. 62/172,294, filed on Jun. 8, 2015.

### FIELD OF THE INVENTION

The invention provides a microfluidic plate for various biological experiments and analyses. In particular, the microfluidic plate comprises one or more microfluidic units formed on or in the plate.

### BACKGROUND OF THE INVENTION

There are several multiple fluid sample processors available at the moment, some of which are micro in size and able to carry out dozens to hundreds of experiments and analyses simultaneously. These devices, often called microfluidic devices, are particularly useful in combinatorial chemistry and DNA synthesis. Microfluidics relates to one or more networks of microscale channels in which a chemical or molecular process or reaction takes place by virtue of fluidic properties at such scale. These devices provide discovery and diagnostic tools which increase the speed and efficiency of discovering new drug candidates and analyzing DNA materials, and do so on a miniaturized scale or platform that reduces cost and manual handling.

For example, chemotherapy is one of the major forms of cancer treatments. The treatment reduces tumor size so that the risk occurred in tumor resection and the risk in recurrence can be eliminated. The most effective regimens including types, doses and administration regiments of anticancer drug are still decided based on general empirical information rather than responses of individual anticancer drug. The empirical information provides the possibility of suitable anticancer drug treatment for specific patients by examining the gene expression of biopsy. Dependency on general empirical data could lead to repetitive and ineffective chemotherapy which can seriously harm patient physically and psychologically. The difference in these indicators could cause dramatic variation in individual anticancer response. In order to achieve more effective treatment, many approaches have been developed. The feasibility of prediction of individual chemotherapy has been shown with in vitro testing using different tumor chemosensitivity assay methods.

For assays with different drugs of multi-concentrations on tumor specimens, the cell-death assay was developed to provide information of cell apoptosis under combinations of drug treatment. For example, differential staining cytotoxicity (DiSC) assay makes it possible to test different drugs of multi-concentrations on fresh tumor specimens. This assay is capable of discriminating drug effects on cancer cells from those on normal cells, which is important since many different types of cells exist in one biopsy. Researchers develop assays not only to find the most effective treatment but also to exclude the less effective treatment. The extreme drug resistance assay (EDR) was developed to this end. The assay eliminates the possibilities of ineffective chemotherapy. On the other hand, in order to develop assays which are processed in environment which is similar to the conditions in a living host, in vivo assays were also developed for prediction of chemosensitivity. Subrenal capsule assay (SRCA) is one of these assays and tests drug response by treating mouse’ kidneys having tumors with the drug. This enables drug response assay in living organism, which may help predict chemosensitivity on the basis of the response from a living host. In addition, histo-culture drug response assay (HDRA) maintains the cell-cell contact as in their native three-dimensional tissue environment so that chemosensitivity assay may be more accurately practiced. The reason to maintain the three-dimensional tissue structure for anticancer drug assay is that cells may response differently to same doses of anticancer drug compared to monolayer culture. The three-dimensional (3-D) tissue culture may increase drug resistance in cancer cells since cells remain in tight connection with each other, which allows them to produce more extracellular matrix. Meanwhile, another assay which studies the expression of genes coding for specific drug-resistance-related enzymes in specimens by applying microarray technology was developed to estimate possibly favorable chemotherapy for specific patients. This assay expects the rate of recurrence after surgery and also the suitable treatment by analyzing the expression of twenty-one genes involved in tumor proliferation and the characteristics reported to be associated with chemotherapy response in general. Many assays were developed and have shown reasonable correlation between in vitro resistance and clinical resistance of the patient's tumor to the same regimen. Roughly, gene expression profiling and cell-based chemosensitivity profiling are used to predict drug response. Some assays were not further developed due to the complexity during operation while some of them have been commercialized and the prediction is proven feasible for clinical use.

In addition to the conventional assays mentioned above, microfluidic technology has also shown feasibility of anticancer drug screening on cell line. Microfluidic drug perfusion system was developed for continuous drug treating environment which provides more in vitro like drug response assay platforms since cells in living organisms are perfused instead of statically treated. Micro 3-D spheroids or clusters are further developed to explore drug resistance of cancer cells in tissues. High content drug screening allows investigation of multi-drug and multi-concentration simultaneously, which can provide much information on drug efficacy enabling a range of treatment options. Although these microfluidic devices have been proven as a possible alternative for anticancer drug assay, much problems still remain unsolved when patients' primary samples are involved.

A common problem being that biopsy specimen is not always sufficient for a complete test to perform combinatorial screening over a range of available drug and their dosage. In order to obtain sufficient number of cells, some conventional assays require days culturing primary cells and a complete test takes 1 to 2 weeks. Thus, a method which is quick and highly accurate and which needs less cells is desirable. U.S. Pat. No. 6,742,661 provides a microfluidic device which conforms with a standard well plate format, including a well plate comprising a plate and an array of wells formed on or in the plate, and a microfluidic structure connecting at least two of the wells. The device can rely exclusively on gravitational and capillary forces that exist in channels within the microfluidic structure when receiving fluid streams. U.S. Pat. No. 6,395,559 provides a method for single well addressability in a sample processor with row and column feeds.

However, these prior art references cannot achieve satisfying effect in drug screening. There is still a need for a microfluidic device for screening drug which is quick, has high throughput, and requires a very small number of cells. The small cell sample requirement for testing is highly desirable since many biopsy cases do not produce large cell sample.

### SUMMARY OF THE INVENTION

The invention relates to a microfluidic plate comprising one or more units, wherein a unit comprising:
(a) a chamber;
(b) one or more fluid channels;
(c) one or more microfluidic gap regions; and
(d) one or more openings;
   wherein the chamber is connected to one or more microfluidic gap regions; a fluid channel is connected to one or more microfluidic gap regions, thereby creating fluidic communication between the chamber and a fluid channel; and one or more openings are connected to a fluid channel. The microfluidic gap creates a region of minimum dimension for fluidic communication between the chamber and a fluid channel wherein the analyte to be tested on the agent (such as cells or organisms) can pass through the microfluidic gap region wherein movement of the agent is prevented due to the height of the microfluidic gap region, thereby retaining the agent (often small cell sample) for testing.

### BRIEF DESCRIPTION OF THE DRAWING

FIG. 1 depicts a microfluidic plate of the invention.

FIG. 2A and FIG. 2B are a perspective view and a diagrammatic cross-section view of a unit, respectively.

FIG. 3A is a top view of a unit of the microfluidic plate of the invention and FIG. 3B is a partial enlarged view of the unit 3.

FIG. 4 shows microfabrication procedures of a unit of the microfluidic plate. (a) Photoresist (SU-8) was patterned on glass substrate to form the microfluidic gap region with etching. (b) Photoresist THB 151N was patterned to form the fluid channel with etching. (c) PDMS was molded over the entire structure. (d) A complete unit was formed after punching holes through the PDMS layer to form a chamber and two openings.

FIG. 5 shows the two-step dilution of cell seeding. The target number of cells in each unit was about 1,000 cells/unit. The original density of cell suspension was first measured with a hemocytometer, hence the dilution ratio needed to arrive at the target cell count can be calculated. The cell suspension was then diluted to obtain the final density of cell suspension. Finally, the density of cell suspension was measured again to determine the corresponding volume for each unit.

FIG. 6 shows the uniformity of cell seeding. The variation on quantity of cells in each unit of the microfluidic plate resulted in the coefficient of variation (CV, standard deviation divided by the mean value) between about 9% to 17%, which is considered acceptable. Although the quantity increased as the original density increased, the CV still remained in an acceptable range.

FIG. 7 shows the operation of solution exchange on 96-well plates and the microfluidic device. (a) The solution exchange conducted on 96-well plate directly introduced and removed solution through the well, which might cause significant cell loss. (b) The solution exchange operated on microfluidic device removed solution from surrounding fluid channels and introduced solution to the center chamber, which should reduce the possibility of contact between cells and tips. The cell loss can be reduced by applying the method stated in (b).

FIG. 8 shows operating procedures of the microfluidic device. I. Cells were suspended in medium and introduced into the chamber with the density of about 1000 cells/unit. II. After overnight incubation, medium was replaced by different concentrations of anticancer drugs. III. After 48 hours of drug treatment, viability was quantified by tri-staining method. The solution was introduced through the chamber and it was removed through the fluid channel.

FIG. 9 shows the correlation between number of live cells and absorbance obtained from MTT assay. The data show that the normalized absorbance increases linearly with the number of living cells. The data were means±standard error of mean (SEM), with each test repeated three times (n=3).

FIG. 10 shows the ability of cell conservation of the microfluidic device after solution exchange. After the solution exchange, the rates of remaining cells in the chamber were shown in this figure, wherein the light black bar represents the rate of remaining cells when the solution was removed from the chamber and the black bar represents the rate of remaining cells when the solution was removed from the fluid channel. When the solution was removed from the chamber directly, the rate of remaining cells was 41.7. When the solution was removed from surrounding fluid channels, the rate of remaining cells increased to 99.2%. (data were mean±standard deviation (SD), n=3; Student's t-test, P<0.01).

FIG. 11 shows toxicity profiles of (a) cisplatin and (b) docetaxel by the invention and on 96-well plates after 24 hour drug treatment on breast cancer cell line MCF7 cells. Cells viability in different units of microfluidic device with drug treatment of different concentrations was normalized against that of untreated cells. No statistical significance in the dose-response between the invention and 96-well plate was found. The dashed line represents the toxicity profile conducted in 96-well plates while the solid line represents that in the invention. The data were means±SEM, n=3: Student's t-test, P<0.05.

FIG. 12 shows toxicity profiles of (a) cisplatin and (b) docetaxel by the invention and on 96-well plates after 24 hour drug treatment on breast cancer cell line MDA-MB-231 cells. Cells viability in different units of microfluidic device with different concentration drug treatment was normalized against that of untreated cells. Although the values of IC50 of cells treated with cisplatin showed differently, no statistical significance in the dose-response between the invention and 96-well plate was found. The dashed line represents the toxicity profile conducted in 96-well plates while solid represents that in the invention. The data were means±SEM, n=3: Student's t-test, P<0.05.

### DETAILED DESCRIPTION OF THE INVENTION

The microfluidic plate of the invention enables high-throughput and rapid in vitro study of the effect of cells under the influence of particular solutions. As microfluidic plate of the invention only requires an extremely low number of cells compared with other well plates or devices. To enable low-cell-number testing, one key feature is the microfluidic gap region which enables analyte to pass through the microfluidic gap region wherein movement of the agent (cells or organisms), immersed within the analyte, is prevented due to the height of the microfluidic gap region, thereby retaining the agent for testing.

Furthermore, since the microfluidic plate can handle extremely low number of cells there is no need for long-term primary culturing before drug test, which means that information of drug response can be obtained more rapidly.

The present invention is more particularly described in the following examples that are intended as illustrative only since numerous modifications and variations will be apparent to those skilled in the art. In the drawings, like numbers indicate like parts throughout the views. As used in the description herein and throughout the claims that follow, the meaning of “a,” “an,” and “the” includes plural reference unless the context clearly dictates otherwise. Also, as used in the description herein and throughout the claims that follow, the meaning of “in” includes “in” and “on” unless the context clearly dictates otherwise.

The term “microfluidic” typically refers to fluids provided to channels having internal dimensions of between 0.1 and 1000 micrometers. While the utilization of fluidic properties in microscale platforms is relatively well-established, enhancements and discovery of new properties are continually being made.

As used herein, the terms “comprising” and “including” or grammatical variants thereof are to be taken as specifying the stated features, integers, steps or components but not precluding the addition of one or more features, integers, steps, components or groups. This term encompasses the terms “consisting of” and “consisting essentially of”.

The invention relates to a microfluidic plate comprising one or more units, wherein a unit comprising:
(a) a chamber;
(b) one or more fluid channels;
(c) one or more microfluidic gap regions; and
(d) one or more openings;
   wherein the chamber is connected to one or more microfluidic gap regions; a fluid channel is connected to one or more microfluidic gap regions, thereby creating fluidic communication between the chamber and a fluid channel; and one or more openings are connected to a fluid channel.

According to the invention, the units can be placed in any configuration, such as linear-linear array or geometric patterns. Typically, units will be arranged in two-dimensional linear arrays on the plate and are each independently formed on or in the plate. The plate can comprise any number of units. Larger number of units or increased unit density can also be easily accommodated using the methods of the invention. In one embodiment, the diameter of the unit ranges from about 1 millimeter to about 40 millimeters. Preferably, the diameter of the unit ranges from about between about 2 millimeters to about 20 millimeters or between about 3 millimeters to about 10 millimeters.

According to the invention, the chamber is where an agent is placed. Preferably, the agent includes various organisms or cells. Organism or cell suspension and/or solution is directly introduced into the chamber and the density of organisms or cells in the chamber is optimized for sufficient interaction. Chamber volume typically can vary depending on chamber depth and cross sectional area. Preferably, the chamber volume is between about 0.1 microliters and about 15 milliliters.

Chambers can be made in any cross sectional shape (in plan view) including square, round, hexagonal, other geometric or non-geometric shapes, and combinations (intra-chamber and inter-chamber) thereof. Preferred are square or round chambers, with flat bottoms.

In one embodiment, the chamber can have a depth between about 0.2 millimeters and about 20 millimeters. Preferably, the chamber depth is between about 0.5 millimeter and about 10 millimeters, more preferably between about 0.5 millimeters to about 5 millimeters or about 2 millimeters and about 8 millimeters and most preferably between about 1 millimeter to 2 millimeters.

In another embodiment, the chambers can have a diameter (when the chambers are circular) or maximal diagonal distance (when the chambers are not circular) between about 0.2 millimeters and about 30 millimeters. Preferably, the chamber diameter is between about 1 millimeter and 30 millimeters or about 1 millimeter and 20 millimeters, more preferably between about 1 millimeter and about 15 millimeters, about 0.2 millimeters and 6 millimeters or between about 1 millimeter and about 10 millimeters, and most preferably, between about 1 millimeter and about 1.8 millimeters.

According to the invention, the chamber is adjacent to and is in fluidic communication with a microfluidic channel. Preferably, the chamber is surrounded with the microfluidic channel. Preferably, the fluid channel is an enclosed channel. In one embodiment, the width of the fluid channel is between about 50 micrometers and about 200 micrometers, preferably between about 0.2 micrometers to about 20 millimeters, between about 0.2 micrometers and about 10 micrometers or between about 5 micrometers to about 5 millimeters, more preferably between about 4 micrometers and about 5 micrometers. The depth of the fluid channel is between about 2 micrometers and about 20 millimeters, more preferably between about 3 micrometers and about 20 millimeters, and most preferably between about 50 micrometers and about 200 micrometers. There are at least one microfluidic gap region between the chamber and the channel and the microfluidic gap region is in fluidic communication with the channel and chamber. The surrounding fluid channel with at least one opening is also applied as reservoir for containing medium and target analyte. Preferably, the target analyte is any analyte to be tested on the organisms or cells in the chamber. Preferably, the target analyte is a drug, agent or environmental pollutant. More preferably, the drug is an anticancer drug.

According to the invention, at least one opening is located at the fluid channel and is in fluidic communication with the microfluidic gap region and the chamber. The opening can be used to remove the solution from and/or introduce the solution into the microfluidic channel. Preferably, at least two openings are located at the channel. In this embodiment, the opening on the channel can reduce bubbles in the units since while introducing solution through one opening, air could be flushed out from the other end. The height of the microfluidic gap region is slightly smaller than that of a single target analyte in the chamber, so the analyte can be conserved in the chamber after solution exchange. Preferably, the height of the microfluidic gap region ranges from about 0.1 micrometer to about 20 micrometers, preferably, about 1 micrometer to about 15 micrometers, and most preferably, about 1 micrometer to about 10 micrometers. The length of the microfluidic gap region is between about 5 micrometers to about 10 millimeters.

The materials for manufacturing the microfluidic plate are preferably polymeric material, thermo-plastic material, glass or quartz, since these materials lend themselves to mass manufacturing techniques. Preferably, the polymeric material is polypropylene, polystyrene, polymethylmethacrylate or polycarbonate. The microfluidic plate can be made of the same or different materials. Preferably, polymers are selected that have low fluorescence and/or high transmittance. Polymeric materials can particularly facilitate plate manufacture by molding methods known in the art or to be developed, such as insert or injection molding or hot embossing. When the material is glass or quartz, the plate can be produced by photolithography or soft lithography. For example, an array of units is cast in Polydimethylsiloxane (PDMS) from masters fabricated by photolithography using photoresist SU-8.

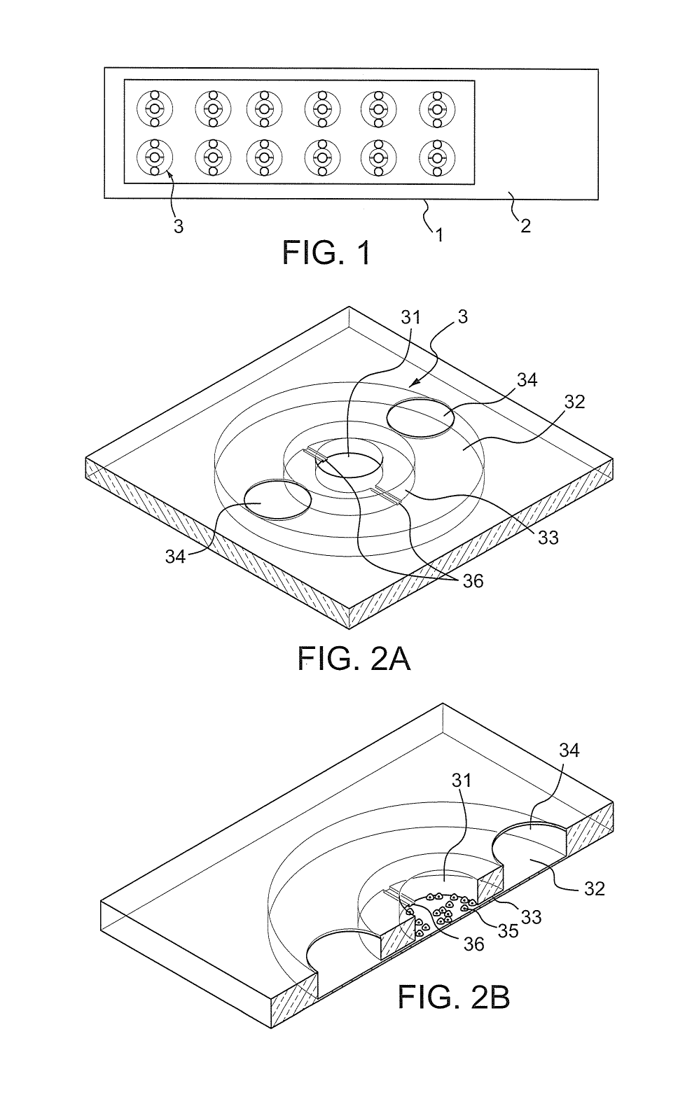

FIG. 1 depicts an overall view of a microfluidic plate 1. The microfluidic plate 1 includes a plate 2 and one or more units 3. The units 3 can be formed on the plate 2, or can be formed on the plate 2 in a separate layer overlying the plate 2.

FIG. 2A and FIG. 2B are a perspective view and a diagrammatic cross-section view of a unit 3, respectively. A unit 3 includes a chamber 31, a fluid channel 32, at least one microfluidic gap region 33 and at least one opening 34. The chamber 31 is the region for cell or organism seeding. Each chamber 31 preferably has a cylindrical or conical shape, or, alternatively, a round shape. The circumference and/or shape of each chamber 31 may also be angular. In one embodiment, the chamber can be used to introduce solution into the unit. The fluid channel 32 surrounds the chamber 31. The channel 32 is designed to contain different analytes that are to be tested on the agent 35 (such as cells or organisms) in the chamber 31. The gaps 33 are between the chamber 31 and the fluid channel 32. The micropillars 36 are formed between the gaps to support the structure. The gaps 33 are for diffusion of analyte into the chamber 31 and for diffusion of waste out to the fluid channel 32. The height of gaps is slightly smaller than the diameter of single cells to prevent cell loss during solution exchange. More preferably, the height of gaps is about 7 micrometers. In one embodiment, at least one opening 34 is located at the channel 32.

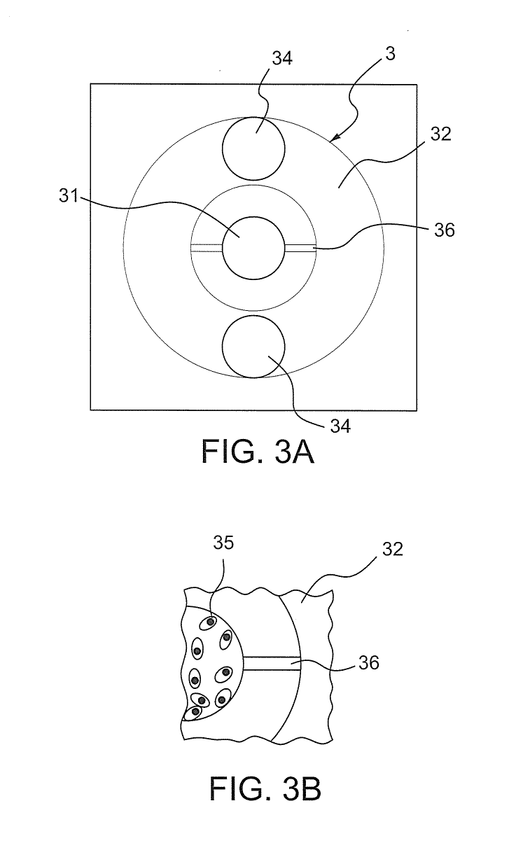

FIG. 3A is the top view of the unit 3. FIG. 3B is a partial enlarged view of the unit 3. The channel 32 is designed to contain an analyte that are to be tested on the agent 35 (such as cells or organisms) in the chamber 31. The openings 34 are located at the channel 32 and are in fluidic communication with the gaps 33. There may be micropillars 36 between the gaps. The openings 34 can be used to remove the solution from and introduce the solution into the channel 32.

In one embodiment, a solution is directly introduced into and removed from the microfluidic plate 1 with pipettes instead of other instruments (i.e. syringe pumps and pressure supplies). This reduces the complexity of the operation, which also provides a user-friendly interface not only for engineers but also those in medical and biological fields. Other complicated and expensive instruments can also be employed. Furthermore, the microfluidic plate 1 enables compatibility with commercial array printers (i.e. SpotArray and Arrayit) for high-throughput and rapid solution introduction and drug treatment.

The unit 3 can be divided into 3 regions: 1. chamber 31, 2. microfluidic gap region 33 and 3. fluid channel 32 with opening 34. One hole punched in the middle of the unit as the chamber 31, while at least one hole punched on fluid channel is opening 34 for removing and introducing solution. In the embodiment with one opening, the solution is introduced through the chamber 31 and is removed from the opening 34. In another embodiment with two or more openings, the solution is introduced through the chamber or one opening and is removed from one of the other openings. The opening on fluid channels can reduce bubbles in units since air will be flushed out from it. The one opening at the middle is the chamber 31 as stated above. The chamber 31 is the region where cells or organisms 35 are plated. Cells or organisms suspension 35 is directly introduced into the chamber 31 and the density of cells or organisms in each chamber is optimized (for example, about 1000 cells/chamber) for sufficient interaction. In addition, the surrounding fluid channel 32 with at least one opening 34 is also applied as reservoir for containing medium and analyte.

In order to prevent direct contact with cells or organisms and prevent cell or organism loss while solution exchange, solution is removed through surrounding fluid channel 32 and introduced into the chamber 31. To easily remove solution from fluid channel 32, chamber 31 and fluid channel 32 are connected by microfluidic gap region 33. During solution exchange, solution in chamber can be made to flow to fluid channel 32 through the microfluidic gap region 33 since unbalanced water level should occur after the solution is removed from the fluid channel 32. The height of microfluidic gap region 33 is slightly smaller than that of a single cell or organism 35 so that cells or organisms 35 can be conserved in chamber 31 after solution exchange.

In the invention, a microfluidic plate is designed for biological experiments and assays. The microfluidic plate needs less cells and less external instruments. The microfluidic plate of the invention presents a user-friendly interface and reduces cell loss. The microfluidic device enables the compatibility with commercial array printers (i.e. SpotArray and Arrayit) for high-throughput and rapid solution introduction and analyte treatment. Cell loss should be reduced on microfluidic plate since the quantity of cells or organisms from each specimen is extremely low compared to that from cell lines. In this invention, the geometry of units on microfluidic plate is optimized to enable low cell loss in every step.

### Example

The following experimental examples are provided in order to demonstrate and further illustrate various aspects of certain embodiments of the present invention and are not to be construed as limiting the scope thereof. In the experiments which follow, the following materials and methods are used:

### 1. Production of Microfluidic Plate of the Invention

### Microfabrication

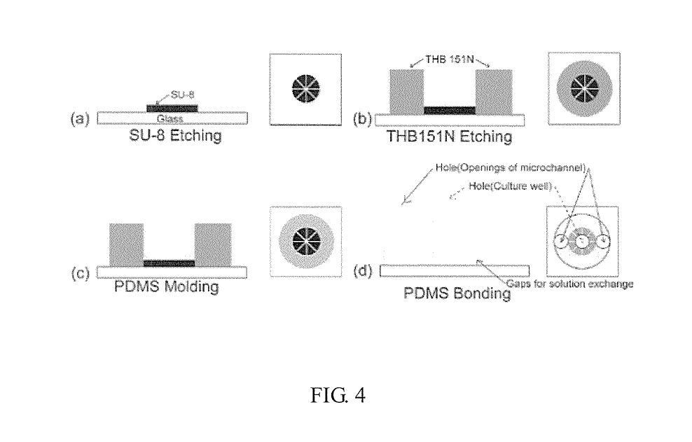

Microfabrication of a collagen-coated microfluidic plate was combined with photolithography, soft lithography, O2 plasma treatment and collagen coating (Duffy, D. C., McDonald, J. C., Schueller, O. J. A. & Whitesides, G. M. Rapid prototyping of microfluidic systems in poly(dimethylsiloxane). Analytical Chemistry 70, 4974-4984 (1998)). An array of units was cast in Polydimethylsiloxane (PDMS) from masters fabricated by photolithography using photoresist SU-8 (GCM 3060, Gersteltec Sarl) and THB 15 1N (THB 15 1N, JSR Micro) on a glass slide. First, SU-8 was micropatterned on glass slide to fabricate the 7 μm high gaps (FIG. 4(a)). THB 151N was then spin-coated on the gaps-patterned glass slide (FIG. 4(b)) for subsequent 100 μm high fluid channel fabrication. After the fabrication of fluid channel, a unit array was formed by PDMS casting as stated above (FIG. 3). Cast PDMS, A:B=1:5 for harder PDMS layer, the unit array was then punched 3 holes on each chamber by stainless tubing to form microunits array. The dimensions of the punched holes were: chamber (1.6 mm) and openings (1.4 mm) After the holes were punched, the PDMS layer was then treated with autoclave for further hardening to prevent collapse at region of gaps since the height of gaps was around 7 μm. The punched PDMS array was bonded onto a conventional lab-use slide after O₂ plasma treatment (60 W, 25 sccm, 570 mTor for 30 sec) to form the final chamber (FIG. 4(d)). Finally, the chamber was plasma-sterilized (50 W, 15 sccm (standard cubic centimeters per minute), 570 mTorr, 4 min) by a plasma generator (CUTE, FEMTO science) (Hsiung, L.-C. et al. Dielectrophoresis-based cellular microarray chip for anticancer drug screening in perfusion microenvironments. Lab on a chip (2011); Mehta, G. et al. Hard Top Soft Bottom Microfluidic Devices for Cell Culture and Chemical Analysis. Analytical Chemistry 81, 3714-3722, (2009)). Collagen (100 μg/mL) (C7661, Sigma-Aldrich) extracted from rat tail was introduced into microfluidic-chamber units to coat the substrates at 37° C. for 1 hour (Madri, J. A. & Williams, S. K. Capillary Endothelial Cell-Cultures—Phenotypic Modulation by Matrix Components. J Cell Biol 97, 153-165 (1983); Folkman, J., Watson, K., Ingber, D. & Hanahan, D. Induction of Angiogenesis during the Transition from Hyperplasia to Neoplasia. Nature 339, 58-61 (1989)).

After the fabrication, the device was stored at 4° C. prior to use. In addition, the device was stored with collagen solution contained in fluid channels and the chambers to prevent bubbles from occurring during storage. The solution in channels of microfluidic device would further decrease the difficulty of first solution introduction.

### Assessment of the Microfluidic Device—Estimation of Cell Seeding Performance

The quantity of cells from each primary sample was significantly lower than that from cell line. As a result, the number of cells introduced into chamber was also significantly smaller than those into wells of 96-well plates due to the need of flow cytometry to analyze the composition of each sample. Since cell behavior could be affected by insufficient interaction between cells, the quantity of cells in each unit may have significant effect on the outcome of dose-response.

### Initial Quantity of Cells

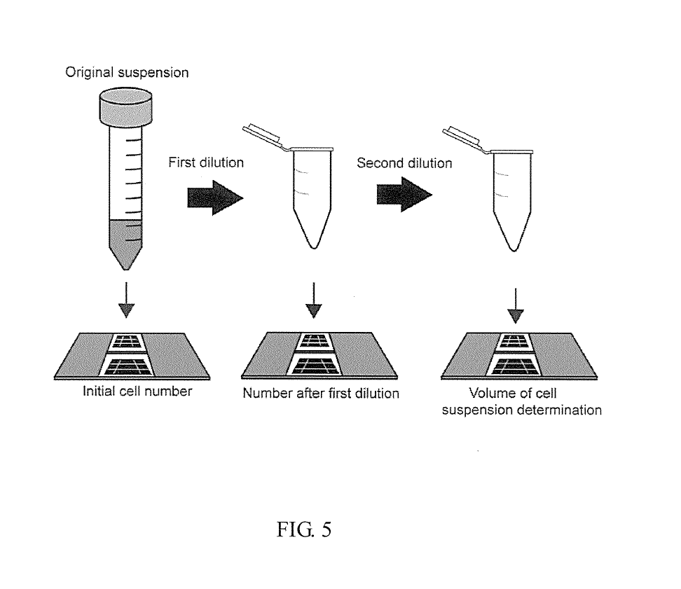

A uniform cell seeding should be obtained for the microfluidic plate since the total amount of cells from each tumor was extremely low and some of the cells needed to be analyzed by flow cytometry to understand the compositions of tissue samples. Approximately, only 10⁵ cells in total could be obtained from one biopsy, which was relatively low compared to quantity of cells from cell lines. In addition, the variation in the cell numbers among units could significantly affect the outcome of tumor response assay since sufficient cell-cell contact would produce more extra-cellular matrix for higher drug resistance (St Croix, B. & Kerbel, R. S. Cell adhesion and drug resistance in cancer. Curr Opin Oncol 9, 549-556 (1997); Desoize, B. & Jardillier, J. C. Multicellular resistance: a paradigm for clinical resistance? Crit Rev Oncol Hemat 36, 193-207 (2000)). To eliminate the variation on the cell numbers between units, a two-step dilution was applied in the preparation of cell suspension to MGP (FIG. 5). The complete operation of the preparation included: 1. measuring the density of original cell suspension; 2. determining how much the cell suspension should be diluted (Ex. N-fold); 3. diluting the cell suspension into N times and then obtaining the expected density after duplicate tests; 4. after the dilution, measuring density again to determine the corresponding volume of cell suspension to each unit. The expected number of cells in each unit was about 1,000 cells/unit. Thus, the optimal volume would then be determined to reach this goal. Theoretically, the larger volume of cells suspension for each droplet, the less variation of cell number.

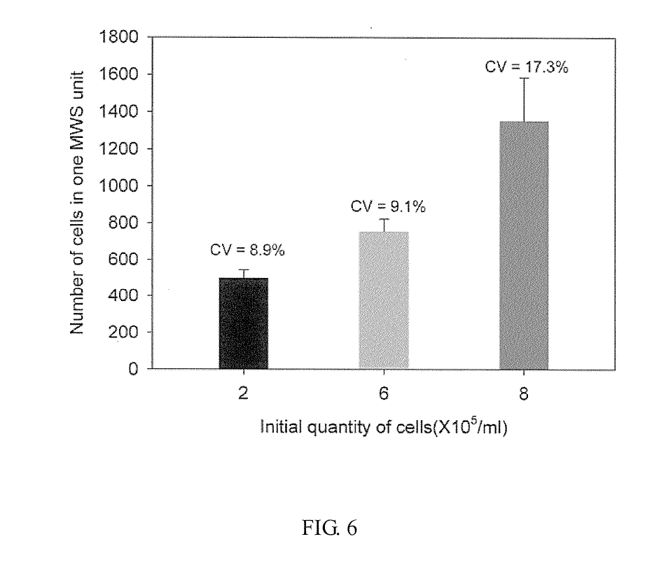

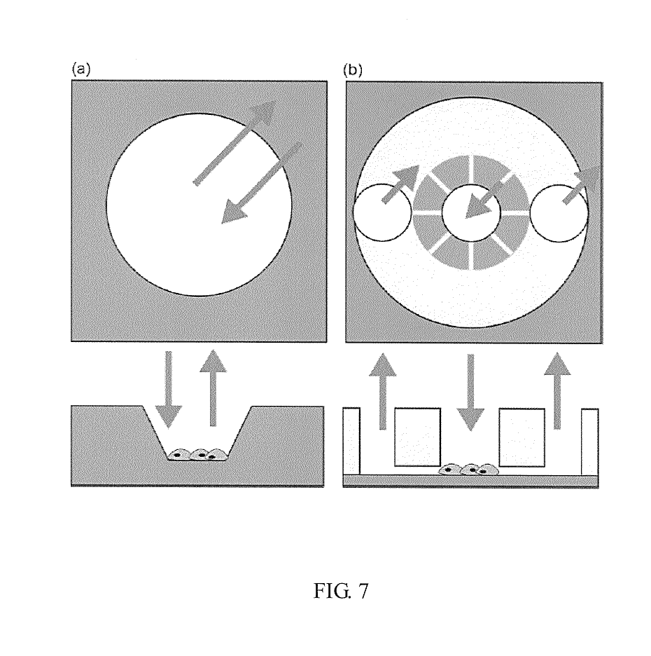

The two-step dilution was more accurate than direct dilution since the concentration of cell suspension was examined after the first dilution, which prevented over-dilution. In order to confirm the reliability of this operation, three different initial densities (2, 6 and 8×105 cells/ml) of cell suspension were randomly chosen and tested. The reason to choose these three densities was that the common densities of primary cell suspension were 10⁵ to 10⁶ cells/ml. The result is shown in FIG. 6.

### Cell Loss Elimination

Cell loss was minimized by removing solution from units of the microfluidic device through surrounding fluid channels instead of directly through the center chamber, which could reduce the contact of tips to solution in microfluidic device (FIG. 7). To confirm that cells were successfully conserved during the operation of the microfluidic plate, the quantities of cells before and after solution exchange were compared. In order to simulate the operation of solution exchange during drug response assay, the operation and duration of cell seeding and solution exchange in this test strictly followed the operation of the microfluidic plate. A fluorescent dye (Hoechst, H 33342) was used to stain cells for better identification. After cells were seeded overnight, fluorescent dye was introduced to identify the initial number of cells. The optimal repeats of solution exchange were conducted as stated in following section. After solution exchange was completed, images were captured to quantify the cells in each unit of microfluidic device. The definition of rate of cells conserved is:

Rate of cells conserved=No. of cells after solution exchange/No. of cells before solution exchange

Difference in quantity of cells was then compared and efficiency of this operation was shown. To further confirm that the optimal operation of solution exchange was removing solution from fluid channels, the rate of remaining cells was compared while removing solution through the chamber and the fluid channel.

### 2. Performance of the Micro-Gap Plate (MGP) on Breast Cancer Cell Lines.

(The Micro-Gap Plate (MGP) is an alias to the microfluidic plate of the invention throughout the context of this invention.)

The anticancer drug screening of cell lines was conducted with MGP and on 96-well plates as control. Two typical breast cancer lines, MCF-7 (ATCC, HTB-22) and MDA-MB-231 (ATCC, HTB-26) were used. The reason to choose the two types of cancer cell lines was that MCF-7 stood for typical luminal type cancer cells while MDA-MB-231 stood for normal-like typically. By using the two cell lines in drug response assay on the microfluidic device, we could examine whether MGP was workable to both the two types of cancer cells since primary samples may have diversely cancer cell types. Anticancer drug screening using cell lines could confirm the feasibility of applying MGP to individualized chemosensitivity assay on primary patient samples. In addition, cell lines on MGP could help optimize the operation of drug response assay beforehand, which would reduce the difficulties when dealing with primary samples. Moreover, the method of viability assessment could be examined on MGP using cell lines since fluorescent images was one of most important factors to determine the viability of cancer cells after drug treatment.

### Preparation of Breast Cancer Cell Lines

Human breast adenocarcinoma cells, MCF7 and MDA-MD-231, were incubated in a culture dish (704001, NEST) at 37° C., 5% CO2. The culture medium were Dulbecco's modified eagle medium with nutrient mixture F12 (DMEM/F12) (12400, GIBCO) for MCF7 cell lines and Dulbecco's modified eagle medium (DMEM) (12100, GIBCO) for MDA-MB-231 cell lines. Both of them were added with 10% fetal bovine serum (FBS) (SV304, Hyclone) and 1% penicillin/streptomycin (15140, GIBCO). While performing drug response, cells were first detached by 0.05% trypsin with EDTA-4Na (tetra-sodium ethylenediaminetetraacetate) (25300054, GIBCO). The detached cell suspension were then diluted into 1000 cells/6 μL by the two-step dilution in the culture medium for subsequent cell seeding in the microfluidic device. In order to fully simulate the drug response assay on primary tumors, the number of cells in each unit of microfluidic device was about 1000. In addition, the procedure of anticancer drug screening in MGP using cell lines were exactly the same as that of drug response assay on patients' samples. Moreover, in order to eliminate the consistent drug response of cells from the same batch, cells for experiments were in different passages but no more than 5 passages in difference.

### Procedure of Anticancer Drug Screening in 96-Well Plates

Anticancer drug screening was also conducted using conventional 96-well plates as controls. Wells of 96-well plates (NEST) were pre-coated with 100 μg/ml of collagen solution for 1 hour. 17000 cells in 100 μl were then seeded into wells for a complete control experiment. The quantity of cells in single well was determined to coordinate the same cell-to-substrate ratio as in MGP (1000 cells/1.766 mm2). After cell seeding, cells were incubated in 37° C., 5% CO, overnight prior to drug treatment. The next morning, culture medium in wells was replaced with anticancer drug-contained medium of different concentrations. Next, cells were incubated in the medium for 24 hours. Afterwards, the bright-field images were captured as the reference. Then, medium was replaced by 12 mM MTT (M5655, Sigma). The MTT treated cells were placed at 37° C. for 3 hours. The MTT assay was based on the cleavage of the yellow tetrazolium salt MTT to purple formazan crystals by dehydrogenases and reductases in living cells 67. The formazan crystals were then solubilized by replacing MTT solution with dimethyl sulfoxide (DMSO) (D4540, Sigma) at 37° C. for 20 minutes. Finally, a plate reader (MQX 200, BioTek) was used to determine the absorbance of the DMSO at 570 nm, which was used to determine the number of living cells. The viability in different concentrations of anticancer drug was normalized against the untreated cells. A correlation between number of living cells and normalized absorbance was built to prove that MTT assay was viable for viability assessment.

### 4. Procedure of Operating the MGP

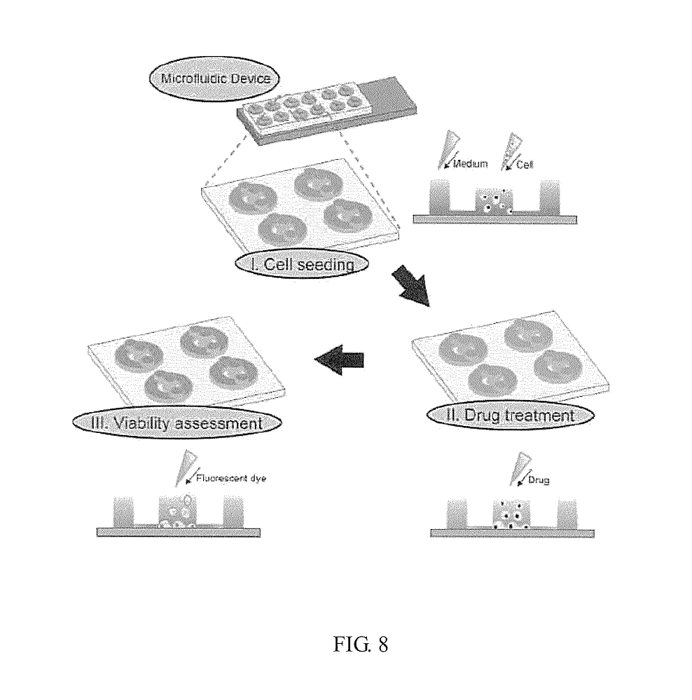

The overall procedure of MGP can be divided into three steps: cell seeding, drug treatment and viability assessment (FIG. 8). All preparations of solution and exchange were done with conventional lab-use pipette (BioPette™).

### Cell Seeding

Primary cells were suspended (16.7 cells/0.1 μL) in culture medium (the formula of culture medium is stated in the previous section). Before cell suspension was into culture chambers, the collagen-contained units of microfluidic device should be washed with culture medium. The cell suspension was then sequentially introduced into 48 chambers (1000 cells/unit). The volume of cell suspension in each unit was determined on the basis of the final density of cell suspension as stated in previous section. 4 slides (48 units) were used (5 drugs×3 concentrations×3 replications).

After cells were successfully introduced into culture chambers, 10 μL of culture medium was then introduced into each fluid channel to create a normal culture environment. The 10 μL of solution to fluid channel would form a curvature of solution at one side the openings, which helped the solution be pumped into fluid channels. Between fluid channel and the chamber were gaps for diffusion of nutrients in culture medium. The height of gaps were extremely low compared to that of fluid channel so that shear stress could be reduced when culture medium and other solution were introduced in subsequent steps since huge shear stress could cause cell detachment. Cells seeded microfluidic devices were individually placed in culture dish (D=10 cm) with sacrificial PBS-filled reservoirs and parafilm sealed to minimize evaporation. The closed microfluidic culture system was incubated at 37° C., 5% CO2 overnight prior to drug treatment.

### Drug Treatment

In this step, different concentrations of drugs were introduced for drug treatment. Two major anticancer drugs, cisplatin (Bristol-Myers Squibb) and docetaxel (Taxotere), were each diluted into 3 concentrations. The culture medium in the microfluidic device was then replaced with the drugs for 48 hours of treatment. The drugs dilutions were replaced with fresh ones every 24 hours to maintain the concentration. In order to prevent bubbles during solution replacement, culture medium in the units of microfluidic device was replaced with repeats of partial replacement. During drug treatment, pictures of treated cells were taken by microscope (Olympus, IX71) every 12 hours.

### 5. Cell Viability Assessment

After 48 hours of drug treatment, viability of cells in each chamber was then determined by tri-fluorescent staining. A fluorescent mixture of PE (conjugated with EpCAM), Hoechst (33342, Invitrogen) and SYTOX (SYTOX®, Invitrogen) was prepared for identification of cell viability. Different drug concentrations were then replaced by fluorescent mixture to stain epithelial cells and dead cells. Bright field images, red fluorescent images (emission of PE, epithelium cells), blue fluorescent (emission of Hoechst, all cells) and green fluorescent (SYTOX, dead cells) of cells images were captured. The cells were automatically counted by journal written in MetaMorph (MetaMorph®) to estimate viability from composed images 74. A correlation between manual quantification and auto quantification by MetaMorph was built to confirm the feasibility of auto cell count. The invention could conserve almost all cells in cultures. Thus, the definition of cell viability is:

Viability=(No. of nucleated cells at 48 h−No. of dead cells at 48 h)/No. of nucleated cells at 48 h

In addition, the number of cells in each chamber was different. The viability of cells treated with different concentrations of anticancer drug was normalized against the untreated cells. Moreover, to determine the IC₅₀ values, the concentration yielding a 50% decrease in normalized percentage cell viability, the dose-response data were treated with a four-parameter logistic equation:

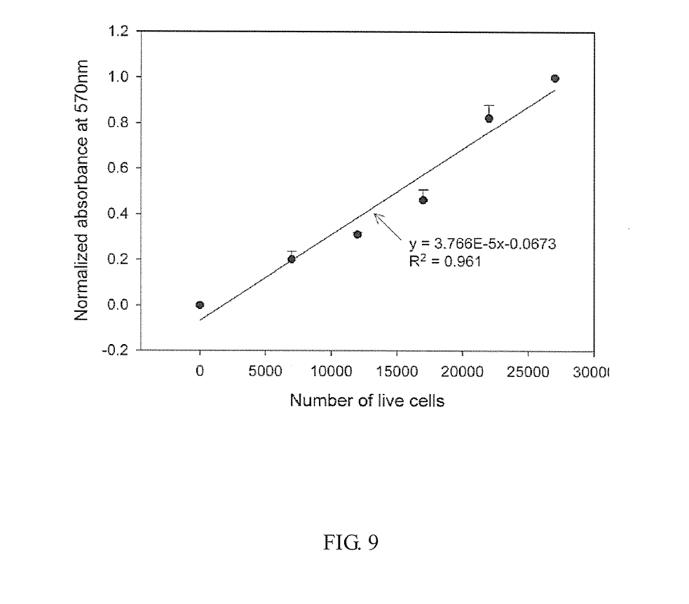

y=yₘᵢₙ+(yₘₐₓ−yₘᵢₙ)/1+10^{(log IC50-x)+curve slope}

where x refers to drug concentration and y refers to normalized viability. The parameters yₘₐₓ, and yₘᵢₙ refer to the upper and lower plateau of the sigmoidal curve of the dose-response, respectively. Curve slope defines the steepness of a toxicity profile. All data of cell viability were presented in means±standard error of the mean (SEM) of the three measurements. Using SEM allowed us to understand the population mean from means of samples studied in the experiments, while SD was regarded as an index of the degree of variability of studied data about the mean of those data 76 (SD was applied to understand the uniformity of cell seeding, stability of solution concentration, cell conservation etc. in this research). On the other hand, the viability of cells after drug treatment in 96-well plates was estimated by applying chamber-accepted MTT assay to measure metabolic function of living cells (FIG. 9).

### 6. Statistical Analysis

Data of at least three engineering related independent experiments were analyzed and values were presented as means±standard deviation (SD) while those of biological related ones were presented as means±standard deviation of the mean (SEM). Student's t-test was applied to statistical significance of specific pairs of the data. Results with a P value less than 0.05 were considered statistically significant. The P values of Student's t-test was obtained with SigmaPlot (Systat Software Inc). Duncan's method was applied to group different approaches of evaporation rate elimination.

### Example 1

### Characterization of the Microfluidic Plate of the Invention Uniform Cell Seeding

In order to retain the cell-cell interaction of primary cells as that in human body, the quantity of cells in each unit of microfluidic device was uniform and as dense as possible. The quantity of cells in each unit was optimized to 1,000 cells/unit and the remaining cells should be sufficient for analysis to understand the ratio of epithelial cells by flow cytometry. By strictly following the process of dilution as stated in previous section (shown in FIG. 5), the quantity of cells in each unit showed good uniformity with CV=9% to 17% (FIG. 6). An important factor to eliminate the variation on cell quantity among units is to quantify the final density of diluted cell suspension for determining the corresponding volume of containing 1,000 cells in each unit. Results in FIG. 6 show that although quantity of cells in each unit was not that identical as expected (1000 cells/unit), the variation among units was eliminated to form a similar cell culture environment between units which would form a more stable cell-based assay platform. The cells in units were quantified by a microscopy automation & image analysis software, MetaMorph.

Cell Remaining after Solution Exchange

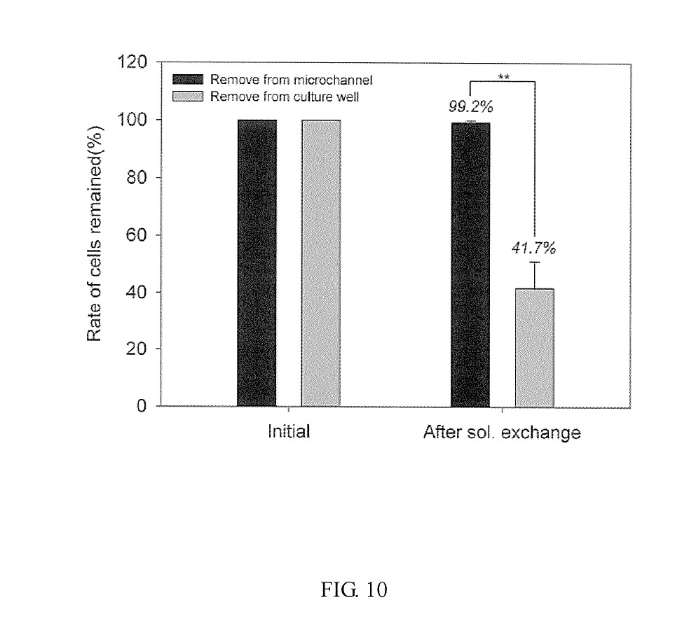

FIG. 10 shows the cell remaining rate during solution exchange and a comparison with directly removing solution from culture well is also shown in the same figure. Gaps in the units of microfluidic device were designed to remove solution through fluid channels instead of directly through culture chambers. This approach can avoid the contact with cells during solution exchange. The solution was directly introduced into culture chamber and the complete operation of solution exchange was stated in previous section. The operation was performed few repeats in order to sufficiently replaced the solution. FIG. 10 shows that after triplicate solution exchanges, cells were conserved in the chamber of units. 99.2% of cells were conserved in the chamber which showed statistical insignificance with the original quantity. On contrast, cells in wells which solution was removed from the chamber directly could be conserved 41.7% which is statistical significance with original quantity. By replacing solution form fluid channels on the units of microfluidic device, cells can be conserved which would have benefit with sufficient cell-cell interaction.

By applying the two-step dilution, the variation on quantity of cells among units was reduced to an acceptable level. In addition, the design of gaps was proven to efficiently reduce cell loss which would assist to maintain cell patterns in the microfluidic device for a complete MGP.

### Example 2

### Chemosensitivity Profiling of Breast Cancer Cell Lines by MGP

MCF7.

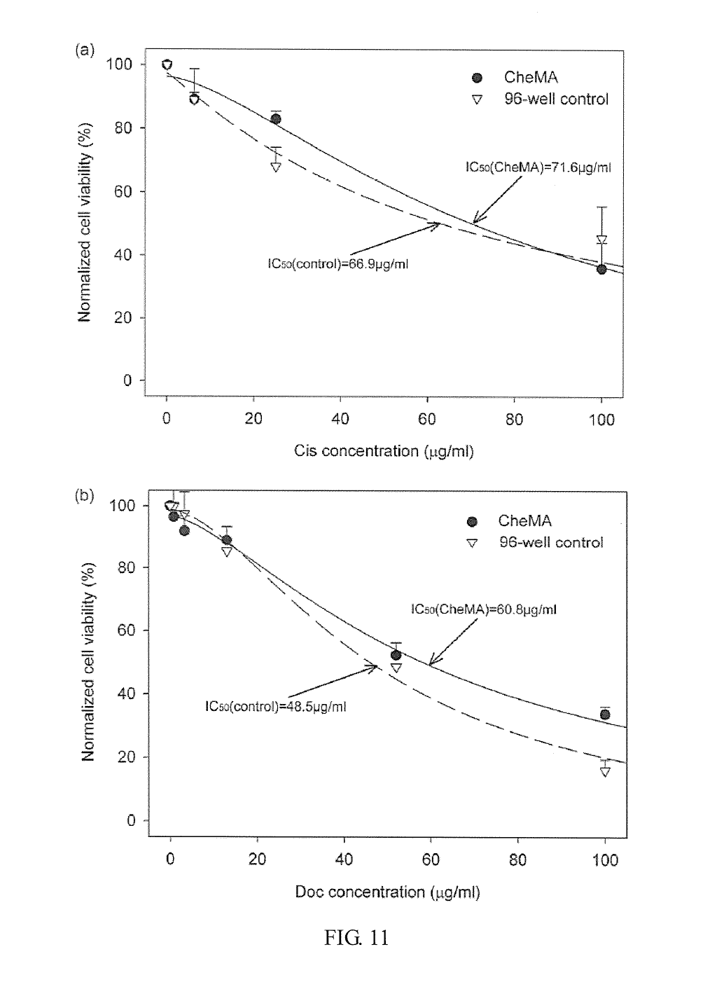

Toxicity profiles of cisplatin and doectaxel by MGP and on 96-well plates obtained after 24 hour drug treatment are presented in FIG. 11. Cell viability of the cells in different units of microfluidic device treated with different concentrations of anticancer drug was normalized against that of untreated cells. By applying Student's t-test, no statistical significance in the dose-response between MGP and 96-well plates was found (P<0.05).

MDA-MB-231.

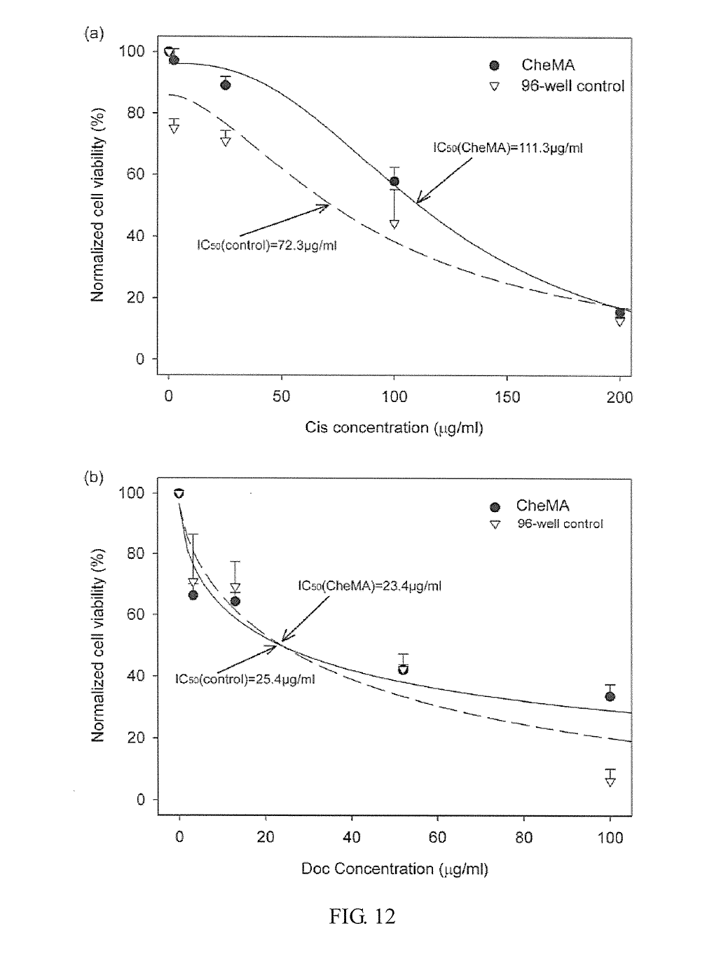

Toxicity profiles of MDA-MB-231 cells treated with cisplatin and docetaxel in different concentrations after 24 hour is presented in FIG. 12. The cell viability in different concentration of anticancer drug was normalized against the untreated ones. The cells were seeded into the microfluidic device and 96-well plates as control respectively. In cells treated with cisplatin, a difference in values of IC₅₀ was found between the platforms. The value of IC₅₀ obtained from MGP was larger than that from 96-well plates. The probably reason may be caused by that a batch of MDA-MB-231 seeded in the microfluidic device grew significantly faster than the other batches. As a result, the number of cells to a specific area in that experiment was found significantly larger than others. Since the intercellular connection would affect drug resistance of cells, the viability after drug treatment of that batch showed significantly higher than others. Although the IC₅₀ values are different between the two platforms in cells treated with cisplatin, by applying Student's t-test, no statistical significance was found between the two. In addition, the values of IC₅₀ of cells treated with docetaxel are similar to each other (23.4 μg/ml in MGP and 25.4 i g/ml in 96-well control). In this case, MGP is further proven to be a workable tool for drug response assay.

### Example 3

### Comparisons Between the Microfluidic Device of the Invention and Conventional Culture Well

To compare with conventional cultures, culture scale and materials, were presented and compared in Microscale cultures in the chip are different from macro cultures in 96-well plate in volume density, surface area to volume ratio and material interactions (Dittrich, P. S. & Manz, A. Lab-on-a-chip: micro fluidics in drug discovery. Nat Rev Drug Discov 5, 210-218, doi:10.1038/nrd1985 (2006)). Microcultures significantly use less volume of reagent which results in higher volume density (more cells in a specific volume of reagent) than in macrocultures. In addition, smaller total volumes of reagents in microcultures cause larger surface area to volume with the materials applied which could further have effect on the cell behavior. When higher surface area to volume ratios are considered with unknown effects of materials during drug response assay, such as the interface between PDMS and reagents, some unsure effects could influence the condition of culture medium, anticancer drug or fluorescent dye. Previous works have suggested that the conventional-used material in microcultures, PDMS, could not be as “inert” as it has been considered (Regehr, K J et al. Biological implications of polydimethylsiloxane-based microfluidic cell culture. Lab Chip 9, 2132-2139, doi:10.1039/b903043c (2009); Toepke, M W & Beebe, D. J PDMS absorption of small molecules and consequences in microfluidic applications. Lab Chip 6, 1484-1486, doi:10.1039/b612140c (2006)) though it has been applied to biological-related microfluidic device many years. Moreover, the PDMS's nature hydrophobic characteristic would further cause more hydrophobic molecules and proteins absorption (Toepke, M W. & Beebe, D. J PDMS absorption of small molecules and consequences in microfluidic applications. Lab chip 6, 1484-1486, doi:10.1039/b612140c (2006); Wang, J. Y et al. Photocatalyzed Surface Modification of Poly(dimethylsiloxane) with Polysaccharides and Assay of Their Protein Adsorption and Cytocompatibility. Analytical Chemistry 82, 6430-6439, doi:10.1021/ac100544x (2010)). Moreover, the higher surface area to volume ratio in microfluidic platforms could lead to more serious absorption. Fortunately, the absorption would be more serious if concentration of solution was generated by fluid channel networks or the solution flew a long distance in fluid channels before experiments. However, in static microfluidic culture system, the molecules of solution would be absorbed into PDMS layer, which may lead to modifying the solution for following procedure (ex. Replace the culture medium by anticancer drug in this research). While the absorption could happen in PDMS-based microculture system, the 96-well plate would cause less uncertainty stated above. In addition, the shifts on concentration of solution would not be an issue since the volume of working solution was relatively high than microcultures. The outcomes of anticancer drug response may be caused by the factors stated above. According to the results conducted on MGP and 96-well control, the toxicity profile of cells treated by different anticancer drugs showed slightly different with each other since the culture environment was different. However, by applying statistical analysis, the results shows no statistical significance between the platforms which proves that the microculture platform is a viable tool for anticancer drug screening.

| | | 96-well plate | | CheMA | |
|---|---|---|---|---|---|
| Difference | | (macroculture) | | (microculture) | |
| Culture | Volume of media | 100 | μl (per well) | 16 | μl (per well) |
| scale | Total surface | 9.2 × 10⁻⁴ | μm⁻¹ (per well) | 2.96 × 10⁻³ | μm⁻¹ (per well) |
| | area/volume ratio | | | | |
| | Surface density | 5.65 × 10⁻⁴ | cells μm⁻² | 5.68 × 10⁻⁴ | cells μm⁻² |
| | Volume density | 170 | cells μl⁻¹ | 62.5 | cells μl⁻¹ |
| | Required cell amount | 17,000 | cells (per well) | 1,000 | cells (per well) |
| Material | PDMS surface | 0 | μm⁻¹ | 2.66 × 10⁻³ | μm⁻¹ |
| | area/volume ratio | | | | |

| | | |
|---|---|---|
| Substrate | Cell culture grade | Collagen-coated glass |
| | polystyrene | |

What is claimed is:

1. A microfluidic plate comprising one or more units, wherein a unit comprising:
(a) a chamber;
(b) one or more fluid channels;
(c) one or more microfluidic gap regions; and
(d) one or more openings;
wherein
the chamber is connected to one or more microfluidic gap regions;
a fluid channel is connected to one or more microfluidic gap regions, thereby creating fluidic communication between the chamber and a fluid channel; and
one or more openings are connected to a fluid channel.

2. The microfluidic plate of claim 1, wherein the height of the microfluidic gap region ranges from about 1 micrometer to about 10 micrometers, thereby creating a region of minimum dimension for fluidic communication between the chamber and a fluid channel.

3. A microfluidic gap region of claim 2, wherein one or more micropillars are located within a microfluidic gap region.

4. The microfluidic plate of claim 1, wherein the chamber is surrounded by one or more microfluidic gap regions.

5. The microfluidic plate of claim 1, wherein one or more microfluidic gap regions are surrounded by one or more fluid channels.

6. A high throughput microfluidic plate comprising at least two of the units of claim 1 arranged in a rectangular array.

7. The microfluidic plate of claim 1, wherein there is one fluid channel.

8. The microfluidic plate of claim 1, wherein the diameter of each unit ranges from about 1 millimeter to about 40 millimeters or between about 2 millimeters to about 20 millimeters or between about 3 millimeters to about 10 millimeters.

9. The microfluidic plate of claim 1, wherein the chamber has a diameter (when the chambers are circular) or maximal diagonal distance (when the chambers are not circular) between about 0.2 millimeters and about 30 millimeters.

10. A microfluidic plate comprising one or more units formed, wherein a unit comprising:
(a) a chamber;
(b) a fluid channel;
(c) one or more microfluidic gap regions; and
(d) one or more openings;
wherein
the chamber is connected to one or more microfluidic gap regions;
the fluid channel is connected to one or more microfluidic gap regions, thereby creating fluidic communication between the chamber and the fluid channel;
one or more openings are connected to a fluid channel;
the chamber is surrounded by one or more microfluidic gap regions; and
one or more microfluidic gap regions are surrounded by the fluid channel.

11. The microfluidic plate of claim 10, wherein the height of the microfluidic gap region ranges from about 1 micrometer to about 10 micrometers, thereby creating a region of minimum dimension for fluidic communication between the chamber and a fluid channel.

12. A high throughput microfluidic plate comprising at least two of the units of claim 10 arranged in a rectangular array.

13. The microfluidic plate of claim 10, wherein the diameter of each unit ranges from about 1 millimeter to about 40 millimeters or between about 2 millimeters to about 20 millimeters or between about 3 millimeters to about 10 millimeters.

14. The microfluidic plate of claim 10, wherein the chamber has a diameter (when the chambers are circular) or maximal diagonal distance (when the chambers are not circular) between about 0.2 millimeters and about 30 millimeters.

15. A microfluidic plate comprises one or more units, wherein a unit comprising:
(a) a chamber;
(b) a fluid channel;
(c) one or more micro fluidic gap regions; and
(d) one or more openings;
wherein
the chamber is connected to one or more microfluidic gap regions along the outer edge or circumference of the chamber;
the fluid channel is connected to one or more microfluidic gap regions along the inner edge or inner radius of the fluid channel, thereby creating fluidic connection between the chamber and the fluid channel; and
one or more openings are connected to the fluid channel.

16. The microfluidic plate of claim 15, wherein the height of the microfluidic gap region ranges from about 1 micrometer to about 10 micrometers, thereby creating a region of minimum dimension for fluidic communication between the chamber and a fluid channel wherein the analyte to be tested on the agent (such as cells or organisms) can pass through the microfluidic gap regions wherein movement of the agent is prevented due to the height of the microfluidic gap region.

17. A high throughput microfluidic plate comprising at least two of the units of claim 15 arranged in a rectangular array.

18. The microfluidic plate of claim 15, wherein the chamber has one opening.

19. The microfluidic plate of claim 15, wherein the diameter of each unit ranges from about 1 millimeter to about 40 millimeters or between about 2 millimeters to about 20 millimeters or between about 3 millimeters to about 10 millimeters.

20. The microfluidic plate of claim 15,
wherein
the height of the microfluidic gap region ranges from about 1 micrometer to about 10 micrometers, thereby creating a region of minimum dimension for fluidic communication between the chamber and a fluid channel wherein the analyte to be tested on the agent (such as cells or organisms) can pass through the microfluidic gap regions wherein movement of the agent is prevented due to the height of the microfluidic gap region;
a high throughput microfluidic plate comprising of 6, 12, 24, 48, 96, 192 or 386 units arranged in a rectangular array;
the diameter of each unit ranges from about 1 millimeter to about 40 millimeters or between about 2 millimeters to about 20 millimeters or between about 3 millimeters to about 10 millimeters, thereby enabling arrangement of units in rectangular array for high throughput purpose; and
the chamber has one opening, thereby enabling agents to be dispensed into and out of the chamber.
